(19) United States
(12) Patent Application Publication
(10) Pub. No.: US 2011/0283374 A1
(43) Pub. Date: Nov. 17, 2011

(54) METHOD FOR PRODUCING HETEROGENOUS EMBRYONIC CHIMERIC ANIMAL USING A STEM CELL

(75) Inventors: Hiromitsu Nakauchi, Tokyo (JP); Toshihiro Kobayashi, Tokyo (JP); Tomoyuki Yamaguchi, Tokyo (JP); Sanae Hamanaka, Tokyo (JP); Masumi Hirabayashi, Okazaki-shi (JP); Megumi Kato, Okazaki-shi (JP)

(73) Assignees: INTER-UNIVERSITY RESEARCH INSTITUTE CORPORATION, MITAKA-SHI, TOKYO (JP); THE UNIVERSITY OF TOKYO, Tokyo (JP)

(21) Appl. No.: 13/146,977

(22) PCT Filed: Jan. 29, 2010
(86) PCT No.: PCT/JP2010/051288

§ 371 Date: Jul. 29, 2011

(30) Foreign Application Priority Data
Jan. 30, 2009 (JP) 2009-020955

(51) Int. Cl. A01K 67/027

(52) U.S. Cl. 800/14

(57) ABSTRACT

A heterologous chimeric animal can be produced by a method comprising the steps of: (A) injecting a stem cell into a blastocyst cavity in a blastocyst stage of an animal heterologous to that of the stem cell, or mixing the stem cell with a divided fertilized egg of the animal heterologous to that of the stem cell; and (B) growing a cell mass including the stem cell prepared in the step (A) into a chimeric animal between a species of the stem cell and a species of the heterologous animal.

### TECHNICAL FIELD

The present invention provides a fundamental technique related to production of a blastular chimeric animal between heterologous animals using a stem cell.

### BACKGROUND ART

When a chimeric animal individual is produced between heterologous animals, generally a so-called aggregation process is adopted in which cell masses during early embryogenesis are mixed. In this process, the following method is adopted. Specifically, each time, fertilized eggs are prepared, and divide to form embryos at a certain stage, which are then mixed with cell masses. Then, the mixture is returned to a surrogate parent, and a litter is expected to be born. As long as such an approach is adopted dependently, a desired chimeric animal is obtained in an extremely complicated manner. Thus, a simpler method for producing a heterologous blastular chimera has been desired.

Non-Patent Literatures 1 to 3 report data on researches for chimera production.

Non-Patent Literature 1 reports establishment of a rat ES cell.

Non-Patent Literature 2 found that the establishment reported in Non-Patent Literature 1 was a mixture of a mouse ES cell, meaning that a blastular chimera of a rat with the mouse ES cell was eventually produced. In other words, it is apparent that these did not intend to produce a heterologous chimeric animal.

Non-Patent Literature 3 states that chimera production is useful in the research on the thymus function. Nevertheless, the chimera is between mice, and hence, the literature is irrelevant to production of a heterologous chimera

### CITATION LIST

### Non Patent Literature

NPL 1: Innaccone P M et al., Development Biology, 1994, vol. 163, pp. 288-292) (Correction in 1997)
NPL 2: Brenin D. et al., Transplantation Proceedings, 1997, vol. 29, pp. 1761-1765
NPL 3: Mueller S. M. et al., PNAS, 2005, vol. 102, no. 30, pp. 10587-10592

### SUMMARY OF INVENTION

### Technical Problems

An object of the present invention is to provide a technique for promptly and readily producing a blastular chimeric animal between heterologous animals. Thus, the present invention aims to provide a fundamental technique that greatly facilitates creation of useful animal species in the field of animal engineering. Moreover, another object is to provide a technique also applicable to a technique for regenerating an “own organ” from a somatic cell, such as skin, depending on the circumstance of an individual. Further, if a technique enabling production of a chimeric animal using an iPS cell is provided, a chimeric animal can be produced from the somatic cell quite readily without destroying an embryo. Thus, the present invention also aims to provide a technique in which an iPS cell can be used in producing a chimeric animal.

### Solutions to Problems

In the present invention, as a result of ernest studies to solve problems in producing a heterologous blastular chimeric animal, it has been discovered that a heterologous blastular chimeric animal can be produced by a method in which a stem cell, such as an ES cell or an iPS cell, is injected into a heterologous-animal blastocyst, or by a method in which the stem cell is mixed with a heterologous-animal fertilized egg having divided several times, and then the mixture is developed. This discovery has led to the completion of the present invention. By use of an ES cell or iPS cell, a heterologous blastular chimeric animal has been successfully produced without preparing a fertilized egg.

Specifically, the present invention provides the followings. (1) A method for producing a chimeric animal, the method comprising the following steps: (A) injecting a stem cell into a blastocyst cavity in a blastocyst stage of an animal heterologous to that of the stem cell, or mixing the stem cell with a divided fertilized egg of the animal heterologous to that of the stem cell; and (B) growing a cell mass including the stem cell prepared in the step (A) into a chimeric animal between a species of the stem cell and a species of the heterologous animal. (2) The method according to the clause, wherein the stem cell is any one of an embryonic stem (ES) cell and an induced pluripotent stem (iPS) cell. (3) The method according to the clause, wherein the stem cell is an iPS cell. (4) The method according to the clause, wherein the iPS cell is reprogrammed using three reprogramming factors of Klf4, Sox2, and Oct3/4. (5) The method according to the clause, wherein the stem cell is an iPS cell, and the mixing is carried out by injection into a blastocyst of the heterologous animal. (6) The method according to the clause, wherein the species of the stem cell is any one of a mouse and a rat. (7) The method according to the clause, wherein the species of the heterologous animal is any one of a mouse and a rat. (8) The method according to the clause, wherein the stem cell is labeled. (9) The method according to the clause, wherein the stem cell is labeled by incorporating a gene coding for a fluorescent protein. (10) The method according to the clause, wherein the stem cell is maintained in a presence of 1000 U/ml or less of a leukemia inhibitory factor (LIF). (11) The method according to the clause, wherein, in the step (A), the stem cell is injected into a center of any one of a blastomere and a perivitelline space of an embryo, or injected near an inner cell mass (ICM) of the blastocyst. (12) The method according to the clause, wherein, in the step (A), a predetermined number of the stem cells appropriate for the chimera formation are injected. (13) The method according to the clause, wherein a medium used in the step (B) is any one of an mR1ECM medium and a KSOM-AA medium. (14) The method according to the clause, wherein the step (B) includes the steps of: returning the cell mixture into a womb of a non-human host mammal that is the heterologous animal; growing the mixture; and thereby obtaining a litter. (15) The method according to the clause, wherein the blastocyst is obtained from any one of a rat four days or later after pregnancy, and an animal in a corresponding stage, and the returning step into the womb is carried out on a rat three days after pseudo-pregnancy or in a corresponding stage. (16) A chimeric animal produced by the method according to any one of clauses 1 to 16. (17) Any one of an organ and a part thereof from a chimeric animal produced by the method according to any one of clauses 1 to 16. (18) A stem cell for producing a chimeric animal having a desired genome type. (19) The stem cell according to clause 16, which is an iPS cell. (20) A method for producing an organ having a desired genome type, the method comprising the following steps: (A) injecting a stem cell of a species having the desired genome type into a blastocyst cavity in a blastocyst stage of an animal heterologous to that of the stem cell, or mixing the stem cell with a divided fertilized egg of the animal heterologous to that of the stem cell; (B) growing a cell mass including the stem cell prepared in the step (A) into a chimeric animal between the species of the stem cell and a species of the heterologous animal; and a (C) extracting an organ having the desired genome type from the chimeric animal.

In the present invention, a non-human animal serving as the origin of the embryo to be developed into the heterologous animal may be any animal other than human, such as pig, rat, mouse, cattle, sheep, goat, horse, dog, chimpanzee, gorilla, orangutan, monkey, marmoset, and bonobo. It is preferable to collect embryos from a non-human animal having a similar adult size to that of the animal species for the stem cell to be mixed.

Meanwhile, a mammal serving as the origin of the stem cell to be transplanted or mixed may be either human or a mammal other than human, such as, for example, pig, rat, mouse, cattle, sheep, goat, horse, dog, chimpanzee, gorilla, orangutan, monkey, marmoset, and bonobo. As for human, when an ethical problem is solved, it is possible to use human under the requirement.

One aspect of the present invention is that the present invention can be successfully carried out with no problem even if the relationship between a recipient embryo and a cell to be transplanted is a heterologous relationship.

As described above, a chimeric animal can be produced by preparing a cell to be transplanted, mixing the cell with a recipient fertilized egg or transplanting the cell into an inner cavity of a blastocyst stage fertilized egg, forming a chimeric cell mass between an inner cell derived from the blastocyst and the transplanted cell in the inner cavity of the blastocyst stage fertilized egg, and growing the cell mass.

The cell mass including the stem cell is transplanted into a uterus of a surrogate parent that is a pseudo-pregnant or pregnant female animal of the species from which the blastocyst stage fertilized egg is derived. The cell mass (for example, blastocyst stage fertilized egg) including the stem cell is developed in the uterus of the surrogate parent to obtain a litter. In this manner, a chimeric animal can be produced. Moreover, a mammal cell-derived target organ can be obtained from this litter.

### ADVANTAGEOUS EFFECTS OF INVENTION

According to the present invention, a technique is provided for promptly and readily producing a blastular chimeric animal between heterologous animals. The present invention successfully produces a heterologous blastular chimeric animal using an ES cell or an iPS cell without preparing a fertilized egg. Thus, the present invention provides a fundamental technique that greatly facilitates creation of useful animal species in the field of animal engineering, improvement in livestock, and so forth. Moreover, the present invention provides a technique also applicable to a technique for regenerating an “own organ” from a somatic cell, such as skin, depending on the circumstance of an individual. When an iPS cell is used, a chimeric animal can be produced from the somatic cell quite readily without destroying an embryo. Furthermore, if the present technique is applied to organ regeneration, an organ wished to be regenerated has totally the same histocompatible antigen as an individual that needs to have the organ. Accordingly, the rejection reaction can be avoided when the organ is transplanted. In this manner, there are many advantages in use of an iPS cell in comparison with use of an ES cell. If the regulation in the ethical aspect is changed in the future allowing application of human ES cell and iPS cell to production of heterologous blastular chimeric animals also, the use of iPS cell has more practical advantages than that of ES cell. For example, it becomes possible to conduct research and development using organs derived from various genomes, the organs being provided by carrying out the present invention by producing an induced pluripotent stem cell (iPS cell) from a cell having a target genome. This can be said to be a technique which was absolutely impossible in the prior art.

### BRIEF DESCRIPTION OF DRAWINGS

FIG. 1 is a fluorescence microphotograph showing production of a mouse/rat chimera. (a) and (b) in the drawing show injection of a mouse ES cell into an 8-cell stage embryo (a) and a blastocyst (b) of a rat, respectively. (c) to (h) show: an E15.5 fetus of a mouse/rat chimera (c, d); a control of E15.5 (e); a neonate (f, g; a GFP-negative litter is a control); and 1 week after birth (h) (that is a control rat fetus but not a chimera). The upper panels are bright-field images, while the lower panels are fluorescence images. These chimeras were derived from the ES cell expressed by DsRed (c) or an iPS cell expressed by GFP (d, f to h).

FIG. 2a is a fluorescence microphotograph showing analysis of the mouse/rat chimera using an embryonic fibroblast. The embryonic fibroblast was established from a litter of the E15.5 mouse/rat chimera produced by the mES cell injection. The upper panel is a bright-field image, while the lower panel is a red fluorescence image.

FIG. 2b is a drawing showing analysis of the mouse/rat chimera using the embryonic fibroblast. Shown are individual populations of rCD54-positive rat-derived cells and DsRed-positive mouse-derived cells in the chimeric embryonic fibroblast.

FIG. 3 is a fluorescence microphotograph showing the contribution of the mouse iPS cells in a rat organ. (a) shows the neonate produced by the miPS injection into the rat embryo. The left panels are bright-field images, while the right panels are green fluorescence images. (b) shows sections of an arm (in the squared line in (a)). One panel on the left side shows one stained with HE, while three panels on the right side show ones immunostained with an anti-GFP antibody (green) and DAPI (cyan; nucleus). The arrows indicate GFP-positive cells in the blood vessel (left) and the skeletal muscle (right). (c) to (f) show images of organs (heart (c), liver (d), pancreas (e) and kidney (f)) of the chimera. In (c) to (f), the upper left panel is a microscope image, the upper right panel is a fluorescence image thereof, the lower left panel is the section stained with HE, and the lower right panel is the section immunostained with the anti-GFP antibody (green) and DAPI (cyan; nucleus).

FIG. 4 (a) is a drawing showing a strategy for establishing GFP mouse-derived iPS cells. After establishment of GFP mouse tail tip fibroblasts (TTF), three factors (reprogramming factors) were introduced into the TFT, and resulting TTF was cultured in an ES cell medium for 25 to 30 days. Then, iPS colonies were picked up, thereby establishing iPS cell lines. (b) shows photographs of the morphology of thus established iPS cells taken by a microscope equipped with a camera. The left shows GFP-iPS cell #2, and the right shows #3. (c) is a fluorescence microphotograph showing measurements of alkaline phosphatase activity. The iPS cells were photographed under a fluorescent microscope, and subjected to staining using an alkaline phosphatase staining kit (Vector Laboratories, Inc., Cat. No. SK-5200). From the left, a bright-field image, a GFP fluorescence image, and alkaline phosphatase staining are shown. (d) is electrophoresis photographs showing identification of the introduced three factors (reprogramming factors) by PCR on genomic DNA. It is the result obtained from PCR performed on the genomic DNA extracted from the iPS cells. From the top, expressions of Klf4, Sox2, Oct3/4, c-Myc, and Myog genes are shown. From the left, results of GFP-iPS cells #2 and #3, Nanog-iPS (for four factors), and ES cell (NC) as a control are shown. At the very right, a result of distilled water is shown. Insertion of the three factors in the iPS cells used in the present invention was confirmed. (e) is electrophoresis photographs showing analysis of an ES cell-specific gene expression pattern in the cells used in the present invention and confirmation of the expression of the introduced genes, using RT-PCR. From the top, expressions of Klf4, Sox2, Oct3/4, c-Myc, Nanog, Rex1, Gapdh genes are shown. At the bottom, a negative control (RT(−)) is shown. As for Klf4, Sox2, and Oct3/4, the expressions were confirmed each for Total RNA and transgenic (Tg). From the left, expressions of GFP-iPS cells #2 and #3, ES cell (NC) as a control, and TTF (negative control) as another control are shown. At the very right, a result of distilled water is shown. (f) is fluorescence microphotographs showing production of a chimeric mouse using the iPS cells. A result of the production of a chimeric mouse is shown, the production being performed by injecting the established iPS cells into a blastocyst obtained from breeding C57BL6 and BDF1 mouse strains. In the upper part, a bright-field image (left) and a GFP fluorescence image (right) of the mouse on embryonic day 13.5 are shown. In the lower part, an image of the mouse in the neonatal period is shown. What denoted by NC is a negative control.

FIG. 5 is a drawing showing the feature of rat iPS cells. (a) is a schematic drawing of the structure of a lentivirus vector used in establishing the rat iPS cells. To express the 3 factors (Oct3/4, Klf4, Sox2) tetracycline-dependently by infection with a single kind of virus (tet-on system), rtTA was expressed under a UbC promoter, while the three factors were expressed under a TRE promoter. Moreover, to label virus-infected cells and the iPS cell line thus established, EGFP was bound at the downstream of rtTA through IRES for expression in the entire body under the UbC promoter. (b) is a fluorescence microphotograph showing the morphology of the established rat iPS cell (rWEi3.3-iPS cell).

FIG. 6 is a drawing showing production of heterologous chimeras between mouse and rat using iPS cells. (a) is a fluorescence microphotograph showing a result of the analysis of the heterologous chimera in the fetal period. Shown are a chimera (on embryonic day 15: top) obtained by injecting a mouse iPS cell into a rat blastocyst and a chimera (on embryonic day 13: bottom) obtained by injecting a rat iPS cell into a mouse blastocyst. The scale bar in the drawing indicates 2 mm. (b) is graphs showing a result of the FACS analysis using an embryonic fibroblast established from the chimera obtained in (a). For both cases, a peak of EGFP positive was observed, and the contribution from the iPS cell was confirmed.

FIG. 7 is drawings proving the production of the heterologous chimera between mouse and rat. (a) is graphs showing a result of the chimerism analysis by FACS using a fetal liver of the heterologous chimera. A liver was collected from an obtained fetus, and blood cells in the fetal liver were stained with CD45 antibodies respectively specific to mouse and rat. Not only did single positive cells exist, but almost all of the injected iPS cell-derived cells expressed EGFP. (b) is a schematic drawing showing the difference in the intron length between exon 2 and exon 4 at the Oct3/4 locus in mouse and rat. (c) is an electrophoresis photograph showing a result of examining the origins of the CD45-positive cells of the mouse and rat. The mouse CD45 or rat CD45-positive cells in the FACS pattern of the chimera in (a) were sorted, genomic DNA was extracted, and the difference in the length was detected by PCR using a common primer between mouse and rat, arrows in (b)). As a positive control, genomic DNAs extracted from CD45-positive cells in peripheral bloods of mouse and rat, respectively, were used.

FIG. 8 is drawings showing a neonate and an adult of the heterologous chimera between mouse and rat. (a), (b) are fluorescence microphotographs showing the neonate of the heterologous chimera between mouse and rat. (a) is of a neonate obtained by injecting the mouse iPS cell into the rat blastocyst, while (b) is of a neonate obtained by injecting the rat iPS cell into the mouse blastocyst, and EGFP fluorescence indicates the iPS cell-derived cell in both cases. Individuals indicated by the arrows indicate non-chimeras of the litter mates. The scale bar in the drawing indicates 10 mm. (c), (d) are photographs showing the adult of the heterologous chimera between mouse and rat. (c) is of a grown individual obtained by injecting the mouse iPS cell (coat color: black) into the rat blastocyst (coat color: white), while (d) is of a grown individual obtained by injecting the rat iPS cell (coat color: white) into the mouse blastocyst (coat color: black), each of which exhibits spotted coat color. (e) is a graph showing the production efficiency of the neonate and the adult of the heterologous chimera. The chimerism rate in adult, the chimerism rate in neonate, non-chimerism rate, the non-implantation or miscarriage rate are shown with the number of transplanted embryos being 100%.

FIG. 9 is fluorescence microphotographs showing a result of the chimerism analysis of the entire body of the neonate of the heterologous chimera between mouse and rat. (a), (c) show the chimerism of the entire body of the neonate of the heterologous chimera between mouse and rat. (a) is of the neonate obtained by injecting the mouse iPS cell into the rat blastocysts, while (c) is of the neonate obtained by injecting the rat iPS cell into the mouse blastocyst, and EGFP fluorescence indicates the iPS cell-derived cell in both cases. The broken lines indicate organs, B denotes a brain, H denotes a heart, Lu denotes a lung, Li denotes a liver, P denotes a pancreas, A denotes an adrenal gland, and K denotes a kidney. (b), (d) show a result of staining tissue sections prepared from representative organs with an anti-EGFP antibody and DAPI. (b) shows the organs taken out from the chimera in (a), while (d) shows the organs taken out from the chimera in (c). The scale bar in the drawing indicates 2 mm in (a), (c), and 100 μm in (b), (d).

### DESCRIPTION OF EMBODIMENTS

Hereinafter, the present invention will be described. It should be understood throughout the present description that expression of a singular form includes the concept of its plurality unless otherwise mentioned. Accordingly, it should be understood that articles (for example, “a,” “an,” “the,” and the like, in English) for a singular form also include the concept of their plurality unless otherwise mentioned. It should also be understood that the terms as used herein have definitions typically used in the art unless otherwise mentioned. Thus, unless otherwise defined, all technical terms and scientific terms as used herein have the same meanings as those generally understood by those skilled in the art to which the present invention pertains. If there is contradiction, the present description (inclusive of the definition) takes precedence. (Chimeric Animal) The term “chimeric animal” as used herein refers to an animal including genome types derived from genomes of two or more animals. The genomes may be allogeneic or heterologous to each other.

The term “stem cell” as used herein refers to any cell having pluripotency. Representative examples thereof include an embryonic stem (ES) cell, an induced pluripotent stem (iPS) cell, an egg cell, a multipotent germ stem cell (mGS cell), an inner cell mass (ICM cell), and the like.

The term “embryonic stem (ES) cell” as used herein is used to have an ordinary meaning in the art, and refers to a cultured cell line established from a blastocyst cell and having multipotency.

The term “induced pluripotent stem (iPS) cell” as used herein refers to a cell in an undifferentiated state by reprogramming the differentiated state of a differentiated cell by a foreign factor (referred to herein as a “reprogramming factor”).

The term “reprogramming factor” as used herein refers to a factor, a factor group, or a member thereof, which are capable of converting a differentiated cell into an undifferentiated cell. Representative examples thereof include Klf4, Sox2, and Oct3/4.

The term “heterologous” as used herein refers to that the species of an animal is different from that of a stem cell to be transplanted. The combinations of rat and mouse, pig and human, and so on are examples of a heterologous combination. In the present description, it is understood that any species of animals should be targeted. In the present invention, an animal heterologous to that of the stem cell is an animal to serve as a host. Accordingly, when a host is intended to be used, the heterologous animal is a non-human.

The term “blastocyst” as used herein is used to have an ordinary meaning in the art, and refers to an embryo whose cleavage stage has been finished in an early development of a mammal. Typically, in a 32-cell stage, the blastocyst is divided into a trophoblast layer surrounding the outer side of a cell aggregate and an inner cell mass (ICM) at the inner side thereof, and a cavity called a blastocoele is formed in the cell aggregate.

The term “blastomere” as used herein is used to have an ordinary meaning in the art, and refers to a morphologically undifferentiated cell formed by cleavage of a fertilized egg mainly between 2-cell stage and blastula stage.

The term “perivitelline space” as used herein is used to have an ordinary meaning in the art, also called an egg-enveloping cavity, and refers to a gap between the surface of an animal egg and a vitelline membrane or a fertilization membrane directly surrounding the egg.

The term “divided fertilized egg” as used herein refers to a fertilized egg having been subjected to cell division. Typically, examples thereof include fertilized eggs in a 4-cell stage, an 8-cell stage, and a 16-cell stage.

The term “injection” as used herein is one that can be achieved by adopting any appropriate means. An example of such means includes the method described in Nagy, A., Gertsenstein, M., Vintersten, K. & Behringer, R. Manipulating the Mouse Embryos. A Laboratory Manual, 3rd ed. (Cold Spring Harbor Laboratory Press, Cold Spring Harbor, N.Y., 2003). A specific example includes the following. For example, using a piezo-actuated micromanipulator (manufactured by Primetech Corporation), pores were carefully created in the zona pellucida under a microscope; then, approximately 10 mES/miPS cells were injected in each of a center of a blastomere or a perivitelline space of an embryo in a case of a 8-cell stage embryo or a morula, and an inner cavity in a case of a blastocyst. Nevertheless, the injection means is not limited thereto.

The term “mixing” as used herein is one that can be achieved by adopting any appropriate means. An example of such means includes the method described in the above document by Nagy, A. et al. A specific example includes the following. For example, two embryos (morulas) from each of which the zona pellucida is removed with Acid Tyrode solution are placed adjacent to each other in the same culture fluid, and thereby the two are allowed to physically adhere to each other. On the next day, if the adhesion occurs, a single mixed blastocyst is formed.

As a method for “growing into a chimeric animal between a species of the stem cell and a species of the heterologous animal” as used herein, any appropriate method can be adopted. Typically, an example of such a method includes a method including returning the cell mixture in a womb of a non-human host mammal that is the heterologous animal, growing the mixture, and thereby obtaining a litter. Nevertheless, the method is not limited thereto. An example of a variety of the method or other methods includes the method described in the above document by Nagy, A. et al. A specific example includes surgically transplanting an embryo mixture into a uterus at an appropriate period after a surrogate parent is bred for pseudo-pregnancy.

The term “label” as used herein may be any factor capable of distinguishing one from the other in a chimeric animal. As long as genes are different from each other, it can be thought that the genes themselves are labels. Nevertheless, generally one capable of distinguishing with simpler recognition means such as visual inspection is used. Examples of such label include: green fluorescent protein (GFP) genes; red fluorescent proteins (RFP); cyan fluorescent proteins (CFP); other fluorescent proteins; LacZ; and the like. One of cells used in a chimeric animal may incorporate a fluorescence protein for specific detection in an expressible state prior to mixing and injection. For example, as a fluorescent protein used for such detection, the sequence of DsRed. T4 (Bevis B. J. and Glick B. S., Nature Biotechnology Vol. 20, p. 83-87, 2002), which is a DsRed genetic mutant, may be designed so as to be expressed in organs of almost the entire body under the control of a CAG promoter (cytomegalovirus enhancer and chicken actin gene promoter), and then be incorporated into a stem cell by electroporation. By performing a fluorescent labeling on a cell to be transplanted, it becomes possible to easily detect from which cell of the chimeric animal each tissue of the produced animal is derived.

The term “leukemia inhibitory factor (LIF)” as used herein refers to a factor discovered as a factor inhibiting the growth of a leukemia cell and inducing differentiation into a macrophage. The LIF is used in cell culturing to maintain the undifferentiated state of an ES cell.

The term “desired genome type” as used herein refers to a genome type desired for producing chimeric animal or organ.

The term “organ” as used herein is used to have an ordinary meaning in the art, and refers to an organ constituting animal body in general or a part thereof. (Method for Producing Chimeric Animal) In one aspect, the present invention provides a method for producing a chimeric animal. This method comprises the following steps: (A) injecting a stem cell into a blastocyst cavity in a blastocyst stage of an animal heterologous to that of the stem cell, or mixing the stem cell with a divided fertilized egg of the animal heterologous to that of the stem cell; and (B) growing a cell mass including the stem cell prepared in the step (A) into a chimeric animal between a species of the stem cell and a species of the heterologous animal. Because of the ethical problem, a human body is excluded from the host.

In the method of the present invention, any approach can be used, as long as the step (A) is carried out in such a manner that the stem cell is mixed with the fertilized egg in the blastocyst stage or the divided fertilized egg. An example of such an approach is the method described in the above document by Nagy, A. et al.

In the method of the present invention, in the step (B), the cell mass prepared in the step (A) may be cultured during a certain period and then subjected to a general fetal development process, or using an approach corresponding thereto, to thereby be grown into a chimeric animal.

In one embodiment, the stem cell used in the present invention is any one of an ES cell and an iPS cell.

In a preferred embodiment, the stem cell used in the present invention is an iPS cell. As the iPS cell, one having a desired genome can be prepared using a somatic cell as the material. Thus, using an iPS cell, a heterologous chimeric animal having a desired genome can be produced. When an iPS cell is used, a chimeric animal can be produced from the somatic cell quite readily without destroying an embryo. Further, if the present technique is applied to organ regeneration, an organ wished to be regenerated has totally the same histocompatible antigen as an individual that needs to have the organ. Accordingly, the rejection reaction can be avoided when the organ is transplanted. In this manner, there are many advantages in use of an iPS cell in comparison with use of an ES cell. If the regulation in the ethical aspect is changed in the future allowing application of human ES cell and iPS cell to production of heterologous blastular chimeric animals also, the use of iPS cell has more practical advantages than that of ES cell. Such iPS cells can be produced and provided by adopting various methods as described in the present description. Alternatively, iPS cells already produced and maintained may be used.

In a preferred embodiment, the iPS cell used in the present invention is reprogrammed using three reprogramming factors of Klf4, Sox2, and Oct3/4. While not wishing to be bound by theory, the reasons why this combination is preferable include, for example, that since c-Myc serving as a cancer gene is not used, no carcinogenesis is observed, and the like. However, the present invention is not limited to such methods. The method for preparing an iPS cell has been greatly diversified. It has been revealed that iPS cells can be established even with a combination of a small-molecule compound with 2 to 3 reprogramming genes, a combination of an enzyme inhibitor with 2 to 3 reprogramming genes, and other factors. It is understood that an iPS cell can be utilized in the present invention by adopting any of these methods.

In a preferred embodiment, the stem cell used in the present invention is an iPS cell, and the mixing step in the present invention is carried out by injection into a blastocyst of the heterologous animal. While not wishing to be bound by theory, the reasons that this combination is preferable include, for example, that since an iPS cell having a desired genome can be prepared using a somatic cell as the material, a heterologous chimeric animal having a desired genome can be produced, and the like.

In one embodiment, the species of the stem cell used in the present invention is any one of a mouse and a rat, but not limited thereto. While not wishing to be bound by theory, the technique for reprogramming a somatic cell has been established through recent establishment of iPS cells in combination with a specific transcription factor. Thereby, application of a similar method even to large-size animal species, such as pig and cattle, other than mouse and monkey, which are considered to be hard to establish or maintain to date, allows the establishment of pluripotent stem cells that can contribute to embryogenesis. These can be used for production of heterologous chimeras. Moreover, since existence of pluripotent stem cells have been recognized in primates such as monkey and human, heterologous chimeras can be produced using the pluripotent stem cells.

In one embodiment, the species of the heterologous animal used in the present invention is any one of a mouse and a rat, but not limited thereto. By the present invention, similar production is possible for large-size animals, such as pig and cattle. The reasons include the followings. While not wishing to be bound by theory, actual production of a heterologous chimera between goat and sheep has been already reported. It has been suggested that chimera production is possible from the level of experimental animals and from heterologous animals having different chromosome numbers, such as mouse and rat presented herein. In addition, even for large-size animals, a chimera individual can be produced between goat and sheep; accordingly, a chimera individual can be produced even between pig and cattle by adopting a method in which a pluripotent stem cell is incorporated inside an embryo as demonstrated in the present case.

In one embodiment, as the stem cell used in the present invention, one that is labeled can be used. As such a label, the stem cell itself may be labeled, or may be modified for labeling. For example, GT3.2 cells used in Example are cells that ubiquitously express an enhanced green fluorescent protein (EGFP) under the control of the CAG expression unit. Moreover, EB3DR cells used in Example are derived from an EB3 ES cell and have a DsRed-T4 gene under the control of the CAG expression unit. Such cells can be said as cells modified to express the label. For other labeling methods, description of other part of the present description can be taken into consideration, or known techniques in the art can be applied.

In one specific embodiment, the stem cell used in the present invention is labeled by incorporating a gene coding for a fluorescent protein (for example, green fluorescent protein).

One embodiment is characterized in that the stem cell used in the present invention is maintained in a presence of 1000 U/ml or less of a leukemia inhibitory factor (LIF). This shows that, although a chimera is produced in Non-Patent Literature 1 using a relatively high concentration LIF as in the prior art, the present invention has revealed that chimerization proceeds with a low concentration LIF.

Examples of preferable LIF concentration include 1000 U/ml or less, 2000 U/ml, 3000 U/ml or less, 500 U/ml, 300 U/ml or less, 200 U/ml or less, 100 U/ml or less, and the like. Examples of the lower limit include 0 U/ml or more, 10 U/ml or more, 20 U/ml or more, 30 U/ml or more, 50 U/ml or more, 100 U/ml or more, 200 U/ml or more, 300 U/ml or more, and the like.

One embodiment is characterized in that, in the step (A) of the present invention, the stem cell is injected into a center of any one of a blastomere and a perivitelline space of an embryo, or injected near an inner cell mass (ICM) of the blastocyst used in the present invention. While not wishing to be bound by theory, it has been revealed that a donor (that is, a mouse in Example 1) is preferably injected into a mass in a host (a rat in Example 1) as deeply as possible in both cases of injection into the blastocyst and of aggregation. While not wishing to be bound by theory, it is conceivable that this is because the donor can thus escape more or less from the immune system of the host.

One embodiment is characterized in that, in the step (A) of the present invention, a predetermined number of the stem cells appropriate for the chimera formation are injected. The predetermined number appropriate for the chimera formation is 1 to 20, preferably 5 to 15, and more preferably 8 to 12.

In one embodiment, a medium used in the step (B) of the present invention is any one of an mR1ECM medium and a KSOM-AA medium. For a mouse, M16, CZB, KSOM, and KSOM-AA that is KSOM with an amino acid being added can be used. Among these, KSOM-AA that provides the best conditions for embryogenesis can be selected.

In one embodiment, the step (B) includes returning the cell mixture into a womb of a non-human host mammal that is the heterologous animal, growing the mixture, and thereby obtaining a litter.

In one embodiment, the step (B) of the present invention is preferably carried out four or five days after fertilization. While not wishing to be bound by theory, it is conceivable that this is because these dates are appropriate in balance between chimera production and immune resistance inhibition. Thus, based on these data, the step (B) can be carried out on pig, cattle, and the like in accordance with the description in the present description.

In one embodiment, it may be advantageous that the blastocyst is obtained from any one of a rat four days or later after pregnancy, and an animal in a corresponding stage, and that the returning step into the womb is carried out on a rat three days after pseudo-pregnancy or in a corresponding stage. Thus, based on these data, the returning step into the womb can be carried out on blastocysts of pig, cattle, and the like in accordance with the description in the present description.

In another aspect, the present invention provides a chimeric animal produced by the method of the present invention. The chimeric animal of the present invention is characterized by being a blastular chimera between heterologous animals, and by using a stem cell such as an ES cell or an iPS cell. Being a chimeric animal can be proved by discrimination of the somatic cell by surface antigen, or by genotyping or searching with a marker gene. (Production of Desired Organ) Another aspect provides a method for producing an organ having a desired genome type, the method comprising the following steps. This method comprises the steps of: (A) injecting a stem cell of a species having the desired genome type into a blastocyst cavity in a blastocyst stage of an animal heterologous to that of the stem cell, or mixing the stem cell with a divided fertilized egg of the animal heterologous to that of the stem cell; (B) growing a cell mass including the stem cell prepared in the step (A) into a chimeric animal between the species of the stem cell and a species of the heterologous animal; and (C) extracting an organ having the desired genome type from the chimeric animal.

The effect of this method can be checked by using known measurement techniques with a marker, an enzyme, a function, and the like specific to each organ.

In the method for producing an organ of the present invention, the organ to be produced may be any solid organ with a fixed shape, such as kidney, heart, pancreas, cerebellum, lung, thyroid gland, hair, and thymus. Preferable examples thereof include kidney, pancreas, hair, and thymus. Such solid organs are produced in the body of a litter by developing totipotent cells or pluripotent cells within an embryo that serves as a recipient. The totipotent cells or pluripotent cells can form all kinds of organs by being developed in an embryo. Accordingly, there is no limitation to the solid organ that can be produced depending on the kind of the totipotent cells or pluripotent cells to be used.

Meanwhile, the present invention is characterized in that an organ derived only from the transplanted cells is formed in the body of a litter individual derived from a non-human embryo that serves as a recipient. Thus, it is not desirable to have a chimeric cell composition of the transplanted cells and the cells derived from the recipient non-human embryo. Therefore, as the recipient non-human embryo, it is desirable to use an embryo derived from an animal which has an abnormality associated with a lack of development of the organ to be produced in a development stage, and whose offspring has a deficiency of the organ. As long as the animal develops such an organ deficiency, a knockout animal having an organ deficiency as a result of the deficiency of a specific gene or a transgenic animal having an organ deficiency as a result of incorporating a specific gene may be used.

For example, when a kidney is produced as the organ, embryos of a Salll knockout animal having an abnormality associated with a lack of development of a kidney in the development stage (Nishinakamura, R. et al., Development, Vol. 128, p. 3105-3115, 2001), or the like, can be used as the recipient non-human embryo. Meanwhile, when a pancreas is produced as the organ, embryos of a Pdx1 knockout animal having an abnormality associated with a lack of development of a pancreas in the development stage (Offield, M. F., et al., Development, Vol. 122, p. 983-995, 1996) can be used as the recipient non-human embryo. When a cerebellum is produced as the organ, embryos of a Wnt-1 (int-1) knockout animal having an abnormality associated with a lack of development of a cerebellum in the development stage (McMahon, A. P. and Bradley, A., Cell, Vol. 62, p. 1073-1085, 1990) can be used as the recipient non-human embryo. When a lung and a thyroid gland are produced as the organ, embryos of a T/ebp knockout animal having an abnormality associated with a lack of development of a lung and a thyroid gland in the development stage (Kimura, S., et al., Genes and Development, Vol. 10, p. 60-69, 1996), or the like, can be used as the recipient non-human embryo. Moreover, embryos of a dominant negative-type transgenic mutant animal model (Celli, G., et al., EMBO J., Vol. 17 pp. 1642-655, 1998) which overexpresses the deficiency of an intracellular domain of fibroblast growth factor (FGF) receptor (FGFR), and which causes deficiencies of multiple organs such as kidney and lung, can be used. Alternatively, nude mice can be used for production of hair or thymus.

(Stem Cell for Producing Chimeric Animal) The present invention provides a stem cell for producing a chimeric animal having a desired genome type. Particularly, the present invention is characterized in that a heterologous chimeric animal can be produced. It is considered that the animal itself is also valuable as an invention because such a chimeric animal could not be produced in the past. While not wishing to be bound by theory, it is conceivable that the reason why such an animal could not be produced in the past is because the success rate is considered to be low from the difficulty in producing a heterologous chimeric animal.

In a preferred embodiment, the stem cell of the present invention is an iPS cell.

(iPS Cells) To produce iPS cells, four factors of Oct3/4, Sox2, Klf4 and c-Myc initially identified may be used, or other methods may also be used. Specifically, iPS cells can be produced when somatic cells are brought into contact with a reprogramming factor (which may be a single factor or in combination of multiple factors) so as to induce initialization. Examples of such initialization and reprogramming factor include the following. For example, by the inventors, in Examples of the present invention, iPS cells were uniquely produced by introducing 3 factors (Klf4, Sox2, and Oct3/4, which are typical “reprogramming factors” used in the present invention) into a fibroblast collected from a tail of a GFP transgenic mouse. Other combinations than this, for example, a method utilizing 4 factors including Oct3/4, Sox2, Klf4, and c-Myc, which are called Yamanaka factors, may also be used, and a modified method thereof may also be used. It is also possible to establish iPS cells using as the gene n-Myc instead of c-Myc, and using as the vector a lentivirus vector, which is a type of retrovirus vector (Blelloch R et al., (2007). Cell Stem Cell 1: 245-247). Further, human iPS cells have been successfully established by introducing 4 genes, which are OCT3/4, SOX2, NANOG, and LIN28, into a fetal lung-derived fibroblast or neonatal foreskin-derived fibroblast (Yu J, et al., (2007). Science 318: 1917-1920).

It is also possible to produce human iPS cells from a fibroblast-like synoviocyte and a neonatal foreskin-derived fibroblast by using human genes homologous to mouse genes, OCT3/4, SOX2, KLF4, and C-MYC, which were used in establishing mouse iPS cells (Takahashi K, et al., (2007). Cell 131: 861-872). It is also possible to establish human iPS cells by using 6 genes which are hTERT and SV40 large T in addition to the four genes including OCT3/4, SOX2, KLF4, and C-MYC (Park I H, et al., (2007). Nature 451: 141-146). Further, although at a low efficiency, establishment of iPS cells in mouse and human by only using 3 factors, Oct-4, Sox2, and Klf4, without introduction of the c-Myc gene has been indicated to be possible. Since the iPS cells are successfully prevented from turning into cancer cells, these can also be used in the present invention (Nakagawa M, et al., (2008). Nat Biotechnol 26: 101-106.; Wering M, et al., (2008). Cell Stem Cell 2: 10-12).

(Knockout Animal) The technique of the present invention can be carried out in combination with a knockout animal. The knockout animal is basically produced according to the following procedure. First, a targeting vector (recombinant DNA) is prepared. Then, by electroporation or the like, the targeting vector is introduced into stem cells (for example, ES cells, iPS cells, or the like). Subsequently, an ES cell line having homologous gene recombination occurred is screened for. Next, the recombinant ES cells, iPS cells, or the like are injected into an embryo in an 8-cell stage or blastocyst stage by the injection method to produce a chimeric embryo. Next, the chimeric embryo is transplanted into a uterus of a pseudo-pregnant animal to obtain a litter (chimeric animal). Next, the chimeric animal thus produced is bred with a wild type animal, and whether or not the germ cell is formed from a cell derived from the recombinant ES cells, iPS cells, or the like is confirmed. Then, animals confirmed to have the germ cells that are formed from the cells derived from the recombinant ES cells, iPS cells, or the like are bred with each other. Litters thus obtained are screened for a knockout animal.
(Gene) In the present description, it is intended that a corresponding gene identified by the electronic search or biological search should also be included in the genes (for example, deficient genes, such as Salll and Pdx-1, or genes necessary for producing an iPS cell, such as Klf4, Sox2, and Oct3/4) used in the present invention.

As used herein, the term “corresponding” gene refers to a gene, in a certain species, which has, or is anticipated to have, an action similar to that of a predetermined gene in a species as a reference for comparison. If there are multiple genes each having such an action, the term refers to a gene having the same evolutionary origin. Therefore, a gene corresponding to a certain gene (for example, Salll) may be an orthologue of the certain gene. Therefore, genes corresponding to human genes may be found in other animals (mouse, rat, pig, rabbit, guinea pig, cattle, sheep, and the like) as well. Such corresponding genes may be identified using a technique that is well known in the art. Therefore, for example, a corresponding gene in a certain animal may be found by searching a sequence database of the animal (for example, mouse, rat, pig, rabbit, guinea pig, cattle, sheep, and the like), using the sequence of a gene, that serves as the reference for the corresponding gene, as a query sequence. (Points to Remember when Using Various Animals) The cases of using animals other than a mouse can be performed by applying a technique described in Examples herein upon paying attention to the following points. For example, regarding the production of a chimera in other species of animals, specifically in a species other than mice, there are more reports of chimeras into which an embryo or an inner cell mass, which is a part of an embryo and is an origin of an ES cell, is injected, rather than of establishment of pluripotent stem cells having an ability to form a chimera (rat: (Mayer, J. R. Jr. & Fretz, H. I. The culture of preimplantation rat embryos and the production of allophenic rats. J. Reprod. Fertil. 39, 1-10 (1974)); cattle: (Brem, G. et al. Production of cattle chimerae through embryo microsurgery. Theriogenology. 23, 182 (1985)); pig: (Kashiwazaki N et al., Production of chimeric pigs by the blastocyst injection method Vet. Rec. 130, 186-187 (1992)). However, even when a chimera into which an inner cell mass is injected is used, the method described herein may be applied. By using an inner cell mass as described above, it is substantially possible to complement a defected organ of a defected animal. In other words, for example, the above-described cells are each cultivated to grow into a blastocyst in vitro, a portion of inner cell mass is physically separated from thus obtained blastocyst, and then, the portion may be injected into a blastocyst. A chimeric embryo can be produced by agglutinating the 8 cell-stage ones or morulas in mid-course. Such a technique is used in production of a heterologous chimeric animal.

(General Techniques) The molecular biological method, the biochemical method, and the microbiological method used in the present description are well known and commonly used in the art, and are disclosed in, for example: Sambrook J. et al. (1989). Molecular Cloning: A Laboratory Manual, Cold Spring Harbor, and its 3rd Ed. (2001); Ausubel, F. M. (1987). Current Protocols in Molecular Biology, Greene Pub. Associates and Wiley-Interscience; Ausubel, F. M. (1989). Short Protocols in Molecular Biology: A Compendium of Methods from Current Protocols in Molecular Biology, Greene Pub. Associates and Wiley-Interscience; Innis, M. A. (1990). PCR Protocols: A Guide to Methods and Applications, Academic Press; Ausubel, F. M. (1992). Short Protocols in Molecular Biology: A Compendium of Methods from Current Protocols in Molecular Biology, Greene Pub. Associates; Ausubel, F. M. (1995). Short Protocols in Molecular Biology: A Compendium of Methods from Current Protocols in Molecular Biology, Greene Pub. Associates; Innis, M. A. et al. (1995). PCR Strategies, Academic Press; Ausubel, F. M. (1999). Short Protocols in Molecular Biology: A Compendium of Methods from Current Protocols in Molecular Biology, Wiley, and annual updates; Sninsky, J. J. et al. (1999). PCR Applications: Protocols for Functional Genomics, Academic Press; separate-volume laboratory medicine “Experimental technique for gene transfer & expression analysis” Yodosha, 1997; and so on. The parts (or could be all) of these documents related to the present description are incorporated herein by reference.

A DNA synthesis technique and nucleic acid chemistry for producing an artificially synthesized gene are disclosed in, for example: Gait, M. J. (1985). Oligonucleotide Synthesis: A Practical Approach, IRL Press; Gait, M. J. (1990). Oligonucleotide Synthesis: A Practical Approach, IRL Press; Eckstein, F. (1991). Oligonucleotides and Analogues: A Practical Approac, IRL Press; Adams, R. L. et al. (1992). The Biochemistry of the Nucleic Acids, Chapman & Hall; Shabarova, Z. et al. (1994). Advanced Organic Chemistry of Nucleic Acids, Weinheim; Blackburn, G. M. et al. (1996). Nucleic Acids in Chemistry and Biology, Oxford University Press; Hermanson, G. T. (1996). Bioconjugate Techniques, Academic Press; and so on. The parts of these documents related to the present description are incorporated herein by reference.

Reference documents cited herein, such as science documents, patents, and patent applications, are incorporated herein by reference in their entirety to an extent that each of which is specifically described.

The preferred embodiments have been described for easy understanding of the present invention. Hereinafter, the present invention will be described based on examples; however, the above description and the following examples are provided only for exemplary purposes and are not provided for the purpose of limiting the present invention. Therefore, the scope of the present invention is not limited to the embodiments or examples which are specifically described herein, and is limited only by the claims.

### EXAMPLES

In the present examples, the following experiments were carried out in compliance with the regulations established in Tokyo University for the handling of animals with the spirit of kindness to animals.

(Reference Documents) In the present examples, the following documents were referenced: Hooper M, Hardy K, Handyside A et al. HPRT-deficient (Lesch-Nyhan) mouse embryos derived from germline colonization by cultured cells. Nature 1987; 326: 292-295. Niwa H, Miyazaki J, Smith A G. Quantitative expression of Oct-3/4 defines differentiation, dedifferentiation or self-renewal of ES cells. Nat Genet 2000; 24: 372-376. Nagy, A., Gertsenstein, M., Vintersten, K. & Behringer, R. Manipulating the Mouse Embryos. A Laboratory Manual, 3rd ed. (Cold Spring Harbor Laboratory Press, Cold Spring Harbor, N.Y., 2003) Qi-Long Ying, Marios Stpyridis, Dean Griffiths, Meng Li, Austin Smith. Conversion of embryonic stem cells into neuroectodermal precursors in adherent monoculture. Nature Biotechnology 2003; 21: 183-186. Shyoso Ogawa, Kahei Satoh, Hajime Hashimoto. In vitro Culture of Rabbit Ova from the Single Cell to the Blastocyst Stage. Nature 1971; 233: 422-424. Oh, S. H., K. Miyoshi H. Funahashi. Rat oocytes fertilized in modified rat 1-cell embryo culture medium containing a high sodium chloride concentration and bovine serum albumin maintain developmental ability to the blastocyst stage. Biology Reproduction 1998; 59: 884-889.

### Example 1

In the present example, it was demonstrated that a chimeric animal was produced through a mixing experiment or injection into a rat fertilized egg or blastocyst using mouse ES cell and iPS cell as a stem cell.

(1) Preparation of Mouse iPS Cell

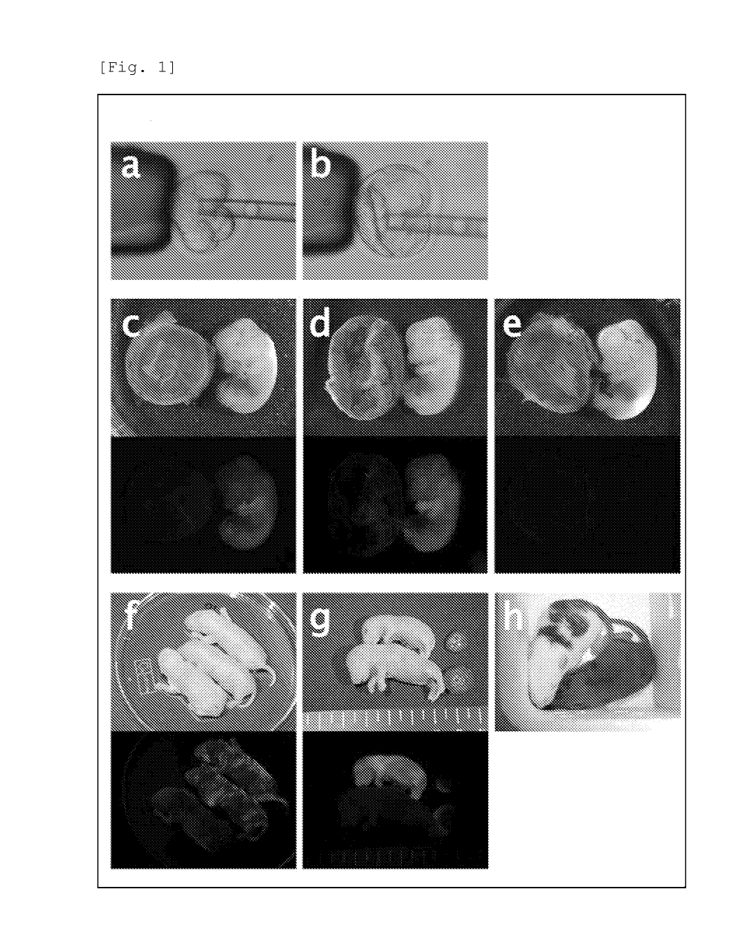

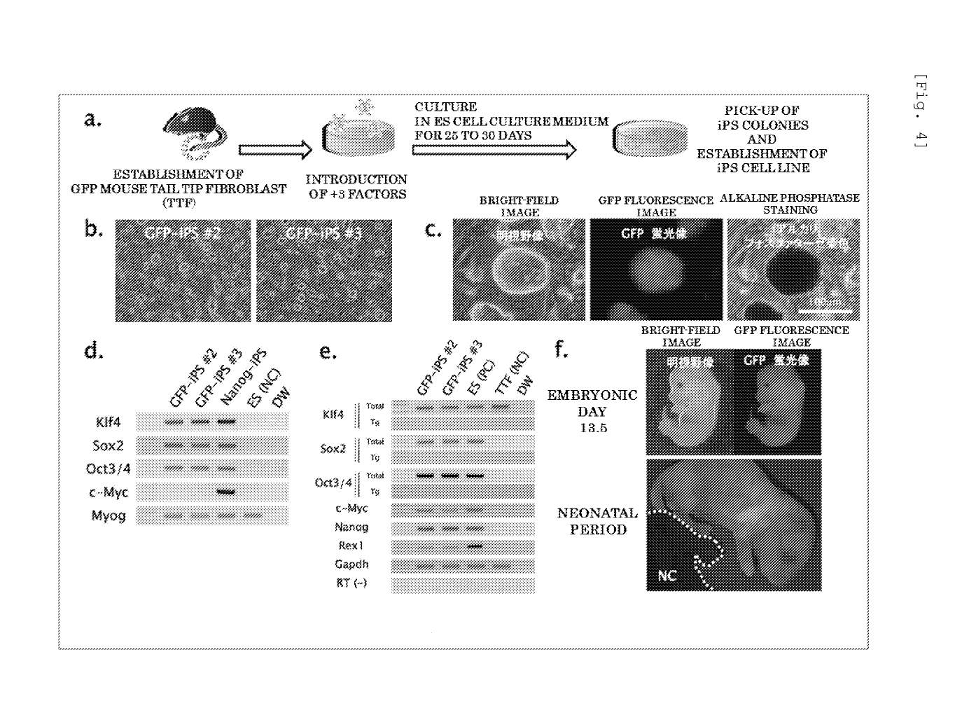

The inventors produced induced pluripotent stem (iPS) cells with 3 factors (Klf4, Sox2, and Oct3/4) by using a fibroblast collected from a tail of a GFP transgenic mouse. The protocol is as follows. The scheme is shown in FIG. 1, and shown in detail in FIG. 4a.

Approximately 1 cm of a tail of a GFP transgenic mouse was collected, peeled, and minced into 2 to 3 pieces. Then, these pieces were placed on MF-start medium (TOYOBO, Japan), and cultured for 5 days. Fibroblasts which appeared there were transferred to a fresh culture dish, and subcultured for several passages to be used as tail tip fibroblasts (TTF).

A supernatant from a virus producing cell line (293gp or 293GPG cell line) produced by introducing a target gene and a virus envelope protein was collected, concentrated by centrifugation, and then frozen for preservation to be used as a virus fluid. The virus fluid was added to a culture fluid of TTF cells which had been subcultured on a previous day to achieve 1×10⁵ cells/6-well plate. This completed the introduction of the 3 factors (reprogramming factors).

On the next day after the introduction of the 3 factors (reprogramming factors), the culture fluid was replaced with a culture fluid for ES cell culture, and the culture was continued for 25 to 30 days. During this, culture fluid was replaced every day.

iPS cell-like colonies appeared after the culture were picked up using a yellow tip (for example, available from Watson), dissociated into single cells in 0.25% trypsin/EDTA (Invitrogen Corp.), and spread on a freshly prepared mouse embryonic fibroblast (MEF).

It was proved that the iPS cell lines established by the above-described method had properties of iPS cells as shown below, that is, being undifferentiated and having totipotency (FIGS. 4b to f).

The morphology of two of thus established iPS cell lines was photographed by a microscope equipped with a camera. After subculturing the iPS cells after the pick-up, they were observed and photographed when they reached the semi-confluent stage on the dish. As a result, it was found that morphologically ES cell-like undifferentiated colonies were formed as shown in FIG. 4b.

Moreover, the iPS cells were photographed under a fluorescent microscope, and subjected to staining using an alkaline phosphatase staining kit (Vector Laboratories, Inc., Cat. No. SK-5200). After observation and photographing of a bright-field image and a GFP fluorescence image by a microscope equipped with a camera, the culture fluid was removed from the iPS cell culture dish, and the dish was washed with a phosphate buffer saline (PBS). Then, a fixing solution containing 10% formalin and 90% methanol was added to the dish, thereby performing a fixing treatment for 1 to 2 minutes. After washing the resultant dish once with a washing solution (0.1 M Tris-HCl (pH 9.5)), a staining solution included in the above kit was added to the dish, and the dish was left to stand in dark for 15 minutes. Thereafter, the dish was again washed with the washing solution, and then observed and photographed. As a result, as shown in FIG. 4c, it was found that, as having been derived from a GFP mouse, the iPS cells produced in the present example constantly expressed GFP, and showed a high level of alkaline phosphatase activity that is characteristic of undifferentiated cells.

Further, for the purpose of identifying the 3 factors inserted into the genomic DNA in the establishment of the iPS cells, the genomic DNA was extracted from the iPS cells and subjected to PCR. The genomic DNA was extracted from 1×10⁶ cells using a DNA mini kit (Qiagen Co., Ltd.) according to the manufacturer's protocol. Using thus extracted DNA as a template, PCR was carried out using the primers below. Oct3/4 Fw (mOct3/4-51120): CCC TGG GGA TGC TGT GAG CCA AGG (SEQ ID NO: 1) Rv (pMX/L3205): CCC TTT TTC TGG AGA CTA AAT AAA (SEQ ID NO: 2), Klf4 Fw (Klf4-51236): GCG AAC TCA CAC AGG CGA GAA ACC (SEQ ID NO: 3) Rv (pMXs-AS3200): TTA TCG TCG ACC ACT GTG CTG CTG (SEQ ID NO: 4), Sox2 Fw (Sox2-5768): GGT TAC CTC TTC CTC CCA CTC CAG (SEQ ID NO: 5) Rv (pMX-AS3200): same as above (SEQ ID NO: 4), c-Myc FW (c-Myc-51093): CAG AGG AGG AAC GAG CTG AAG CGC (SEQ ID NO: 6) Rv (pMX-AS3200): same as above (SEQ ID NO: 4). As a result, the insertion of the 3 factors was confirmed as shown in FIG. 4d.

In addition, a gene expression pattern unique to ES cell and expression of introduced genes were confirmed by reverse-transcription polymerase chain reaction (RT-PCR). 1×10⁵ GFP-positive cells were sorted into Trizol-LS Reagent (Invitrogen Corp.) using a flow cytometer, mRNA was extracted from the cells, and cDNA was synthesized from the mRNA using ThermoScript RT-PCR System kit (Invitrogen Corp.) according to the attached protocol. Thus synthesized cDNA was used as a template to perform PCR. In regard to primers used, the same primers as those used in d above were used for transgene expression (notated as Tg in the drawing), while primers synthesized based on the report by Takahashi K & Yamanaka S (Cell 2006 Aug. 25; 126 (4): 652-5.) or the like were used for other gene expression. As a result, as shown in FIG. 4e, all the lines showed expression patterns approximately the same as that of ES cell. Further, it was found that expression of the introduced gene (Tg) was inhibited by a high level of gene silencing activity of the iPS cell.

Furthermore, thus established iPS cells were injected into a blastocyst to produce a chimeric mouse. Using an ovum collected from a BDF1 strain mouse (female, 8 weeks old) having been subjected to an ovarian hyperstimulation treatment by administration of PMSG and hCG hormones and a C57BL/6-derived sperm, in vitro fertilization (IVF) was performed to obtain a fertilized egg. The fertilized egg thus obtained was cultured to the 8 cell-stage/morula, then frozen for preservation, and recovered the day before blastocyst injection. In regard to the iPS cells, those reached the semi-confluent stage were detached using 0.25% Trypsin/EDTA, and suspended in ES-cell culture media to be used for injection. Blastocyst injection was performed, in the same manner as the technique used for the blastocyst complementation, under a microscope using a micromanipulator. Going through culture after the injection, transplantation into the uterus of an ICR strain surrogate parent was performed. In analysis, observation and photographing were carried out under a fluorescence stereoscopic microscope on embryonic day 13 and postnatal day 1. As a result, as shown in FIG. 4f, iPS cell-derived cells (GFP positive) were confirmed in the fetal period and the neonatal period. Accordingly, it was suggested that the established iPS cell line possessed high multipotency.

(2) Culture of mES/miPS Cell

Undifferentiated mouse embryonic stem (mES) cells (EB3DR) (which were donated by Professor Niwa Hitoshi at RIKEN CDB) were placed on a gelatin-coated dish and maintained in a Glasgow's modified Eagle's medium (GMEM; Sigma Corporation, St. Louis, Mo.) without feeder cells, the GMEM being supplemented with 10% fetal bovine serum (FBS; from NICHIREI Biosciences Inc.), 0.1 mM 2-mercaptoethanol (Invitrogen Corp., San Diego, Calif.), 0.1 mM non-essential amino acid (Invitrogen Corp.), 1 mM sodium pyruvate (Invitrogen Corp.), 1% L-glutamine penicillin streptomycin (Sigma Corporation), and 1000 U/ml leukemia inhibitory factor (LIF; Millipore, Bedford, Mass.). The EB3DR cells are derived from an EB3 ES cell, and carry a DsRed-T4 gene under the control of a CAG expression unit. The EB3 ES cell is a subline cell derived from E14tg2a ES cells (Hooper M. et al., 1987), and established by targeting, on Oct-3/4 allele, the incorporation of an Oct-3/4-IRES-BSD-pA vector constructed so as to express blasticidin, which is a drug-resistance gene, under the control of an Oct-3/4 promoter (Niwa H. et al., 2000).

Undifferentiated mouse induced pluripotent stem (miPS) cells (GT3.2) were maintained on a mitomycin C-treated mouse embryonic fibroblast (MEF) in Dulbecco's modified Eagle's medium (DMEM; Invitrogen Corp.) supplemented with 15% knockout serum replacement (KSR; Invitrogen Corp.), 0.1 mM 2-mercaptoethanol (Invitrogen Corp.), 0.1 mM. non-essential amino acid (Invitrogen Corp.), 1 mM HEPES buffer solution (Invitrogen Corp.), 1% L-glutamine penicillin streptomycin (Sigma Corporation), and 1000 U/ml leukemia inhibitory factor (LIF; Millipore). The GT3.2 cells are cells established from a fibroblast collected from a tail of a male GFP transgenic mouse (donated by Professor Okabe Masaru at Osaka University) into which 3 reprogramming factors, Klf4, Sox2, Oct3/4, are introduced with a retrovirus vector. The GT3.2 cells ubiquitously express an enhanced green fluorescent protein (EGFP) under the control of the CAG expression unit.

(3) Preparation of Mouse 8-Cell/Morula or Blastocyst

These embryos were prepared according to the published protocol (Nagy A. et al., 2003). In a brief description, mouse 8-cell/morula embryos were collected from the oviduct and uterus of a female BDF1 mouse on day 2.5 after outcrossing with a male C57BL/6 mouse into M2 medium (Millipore). These embryos were transferred into drops of KSOM-AA medium (Millipore), and cultured to the blastocyst stage for 24 hours.

(4) Preparation of Rat 8-Cell/Morula or Blastocyst

Rat 8-cell/morula embryos from the oviduct and uterus of a female Wistar strain rat on day 3.5 after crossing with a male Wistar strain rat (herein may also be referred to as 3.5 dpc) and rat blastocysts from the uterus of a female Wistar strain rat on day 4.5 after the crossing (herein may also be referred to as 4.5 dpc) were each collected into a HERs medium containing A solution (HAM F-12 (SIGMA CORPORATION) 1.272 g, NaHCO₃ (SIGMA CORPORATION) (0.192 g)+B solution (RPMI1640 (SIGMA CORPORATION) 0.416 g, NaHCO₃ (SIGMA CORPORATION) 0.056 g)+C solution (EARLE (SIGMA CORPORATION) 0.344 g, EAGLE MEM (SIGMA CORPORATION) 0.0352 g, NaHCO3 0.064 g)+Penicillin G (SIGMA CORPORATION) 0.015 g, and Streptomycin (SIGMA CORPORATION) 0.010 g. These embryos were transferred into a modified rat 1-cell embryo culture medium containing 80 mM NaCl (manufactured by Wako Pure Chemical Industries, Ltd.) and 0.1% polyvinyl alcohol (PVA; Sigma Corporation) (mR1ECM; Oh et al., 1998), and cultured to the blastocyst stage for 24 hours.

(5) Injection of mES/miPS Cell into Rat 8-Cell/Morula or Blastocyst Stage Embryo

The mES/miPS cells were treated with trypsin, and then suspended in the culture medium to which 1 mM HEPES buffer solution had been added. At the 8-cell/morula stage, the embryos were transferred into the fine drops containing HEPES buffer mES/miPS culture medium. Using a piezo-actuated micromanipulator (manufactured by Primetech Corporation), pores were carefully created in the zona pellucida under a microscope. Then, 10 mES/miPS cells were injected into a center of any one of a blastomere and a perivitelline space of each embryo. After the injection, the embryo was cultured to the blastocyst stage in an mR1ECM medium for 24 hours, and thereafter transplanted into the uterus of a surrogate parent female Wistar strain rat on 3.5 dpc bred for pseudo-pregnancy.

At the blastocyst stage, these embryos were transferred into the same fine drops. Then, 10 mES/miPS cells were injected into the blastocyst cavity near an inner cell mass (ICM). After the injection, the embryo was cultured in an mR1ECM medium for 1 to 2 hours, and thereafter transplanted into a surrogate parent. As a control, mES/miPS cells were injected into mouse embryos by the same method. After the injection, these embryos were cultured in a KSOM-AA medium to the blastocyst stage, and thereafter transplanted into the uterus of a surrogate parent female ICR mouse on 2.5 dpc bred for pseudo-pregnancy. The percentage of litters born and the percentage of chimera formation after the transplantation are shown in Table 1.

TABLE 1
Mouse/rat chimera production efficiency

| | | Number of | Number | Number | | | |
|---|---|---|---|---|---|---|---|
| Injected | Stage of | embryo | of | of | Analyzed stage | | |
| cell | embryo | transplanted | litters (%) | chimeras (%) | fetus | neonate | adult |
| mES cell | 8-cell/morula | 85 | 6 (7) | 2 (7) | 2 | — | — |
| (EB3DR) | blastocyst | 59 | 29 (49) | 2 (3) | 2 | — | — |
| miPS cell | 8-cell/morual | 29 | 1 (3) | 0 (0) | — | — | — |
| (GT3.2) | blastocyst | 37 | 22 (59) | 9 (24) | 2 | 3 | 4 |

(6) Analysis of Chimeras

The chimeras were analyzed in the fetal period of 15.5 to 16.5 days, the neonatal period and the adult. An allocation of each analysis period is shown in Table 1. In the analysis in the fetal period, laparotomy was performed on day 12 after the transplantation into the surrogate parent, the fetus was taken out, and the presence of chimera and its contributing proportion were examined with the naked eyes using fluorescences of DsRed in case of the ES cell and EGFP in case of the iPS cell as markers under a fluorescence stereoscopic microscope (Table 1). In the analysis in the neonatal period, a litter was obtained by natural delivery or Caesarean section on day 18 after the transplantation that is in the full term pregnancy, and observed with the naked eyes using the fluorescences as in the fetal period.

(7) Analysis of Embryonic Fibroblast by Flow Cytometry

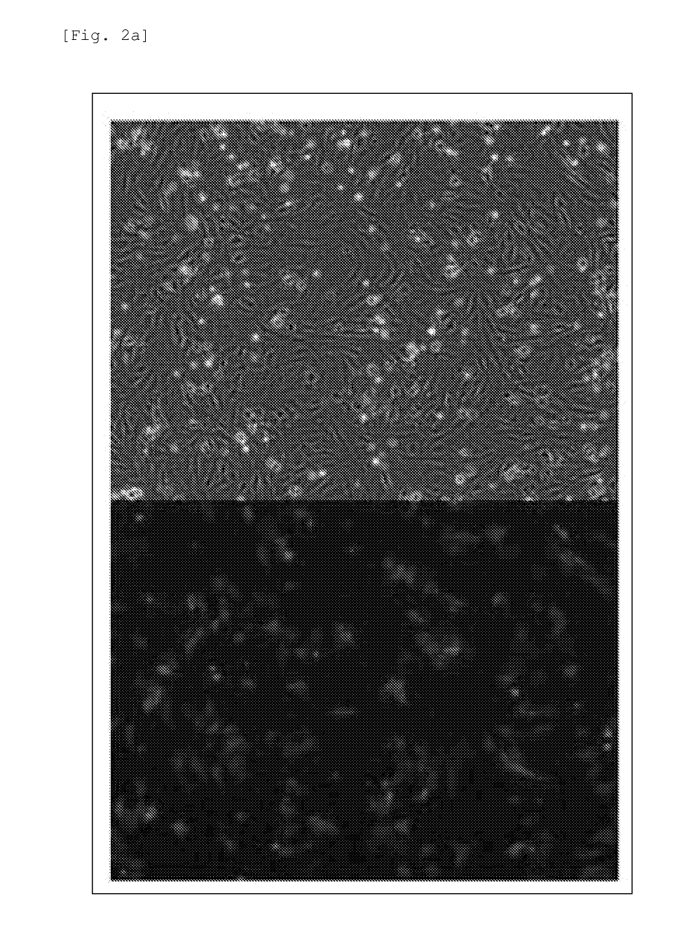

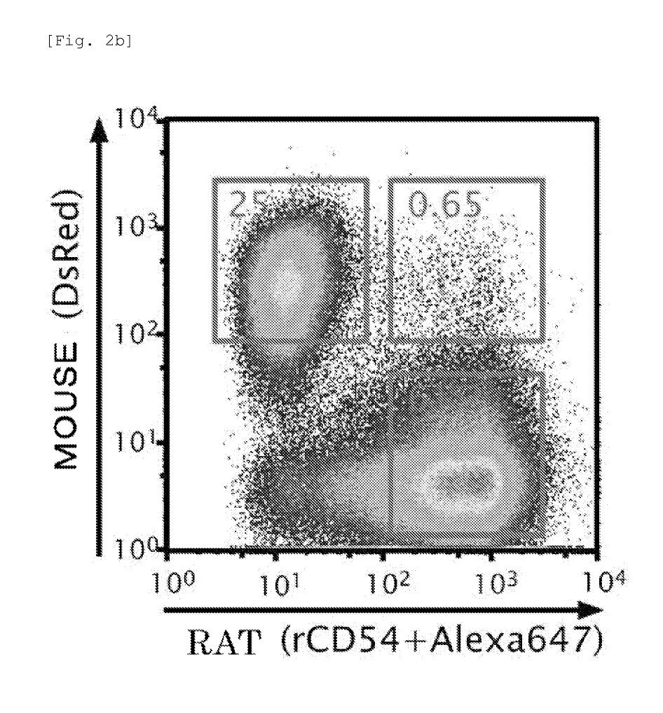

An embryonic fibroblast was established from the litter on day 11 or day 12 after the embryo transplantation (FIG. 2a). Specifically, the fetus was dissected under a microscope, and the head and an organ were extracted, which were then treated with trypsin for 20 minutes. After the trypsin treatment, the suspended material was passed through a filter, and thereafter spread on a gelatin-coated dish. After the incubation for 24 hours, adhered cells were collected. FIG. 2a shows the embryonic fibroblast established from the litter of the E15.5 mouse/rat chimera produced by the mES cell injection, in the analysis of the mouse/rat chimeras using the embryonic fibroblast. The upper panel in FIG. 2a is a bright-field image, while the lower panel in FIG. 2a is a red fluorescence image. Then, the cells were suspended in a phosphate buffer saline (PBS) (staining medium; SM) containing 3% FBS, and subsequently immunostaining was carried out on ice for 1 hour using an anti-rat CD54 antibody (BD Pharmingen, San Diego, Calif.) to which fluorescein isothiocyanate (FITC) was bound. After washing with SM, an anti-mouse IgG antibody (Invitrogen Corp.) to which Alexa647 was bound was added to the cell suspended material in order to enhance the fluorescence intensity, and immunostaining was carried out on ice for 1 hour. After suspended in SM containing propidium iodide (PI), these cells were analyzed by MoFlo (Dako, Carpinteria, Calif.) and Flow-Jo software for distributions of ES-cell derived mouse cells (DsRed) and rat cells (rCD54+Alexa647). FIG. 2b shows the result.

FIG. 2b shows individual populations of rCD54-positive rat-derived cells and DsRed-positive mouse-derived cells of the chimeric embryonic fibroblast, in the analysis of the mouse/rat chimeras using the embryonic fibroblast.

(8) Immunohistochemistry of Mouse/Rat Chimeric Organ and Tissue

Organs and tissues (arm, heart, liver, pancreas, and kidney) from the mouse/rat chimeric neonate were fixed with 4% paraformaldehyde for 6 hours, and embedded in a paraffin. The embedded organs were sectioned, and immunostaining was carried out at room temperature for 1 hour using an anti-GFP antibody (Invitrogen Corp.). After washing with PBS, these sections were immunostained at room temperature for 1 hour using an anti-rabbit IgG antibody (Invitrogen Corp.) to which Alexa488 was bound. The stained sections were covered with a mounting solution containing DAPI (Vector Laboratories, Inc., Burlingame, Calif.), and analyzed under a fluorescence stereoscopic microscope. The sections were also stained with haematoxylin and eosin (HE) and analyzed under an optical microscope.

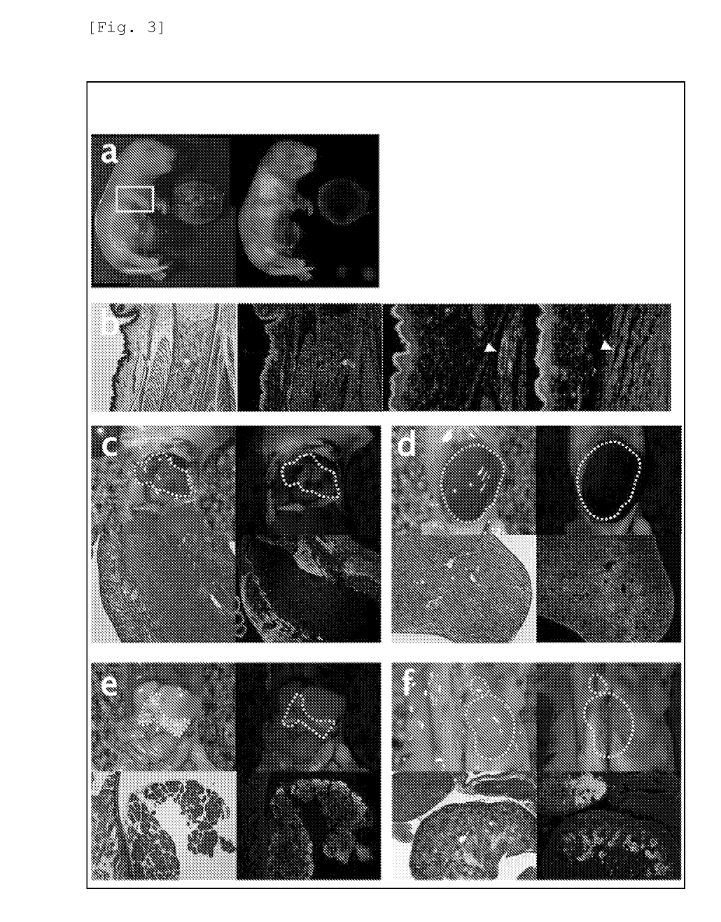

FIG. 3 shows the result. As apparent from FIG. 3, the contribution of the mouse iPS cell in the rat organs is shown. a. shows the neonate produced by the miPS injection into the rat embryo. The left panels are bright-field images, while the right panels are green fluorescence images. b. shows sections of an arm (in the squared line in a.). One panel on the left side shows one stained with HE, while three panels on the right side show ones immunostained with the anti-GFP antibody (green) and DAPI (cyan; nucleus). The arrows indicate GFP-positive cells in the blood vessel (left) and the skeletal muscle (right). c to f. show images of organs (heart (c), liver (d), pancreas (e) and kidney (f)) of the chimera. In c to f, the upper left panel is a microscope image, the upper right panel is a fluorescence image thereof, the lower left panel is the section stained with HE, and the lower right panel is the section immunostained with the anti-GFP antibody (green) and DAPI (cyan; nucleus).

From the above results, the following can be said. Specifically, the mouse iPS cell contributed in the entire body of the rat, suggesting playing a role in the body formation.

### Example 2

To demonstrate the production of a chimeric animal between mouse and rat, both the method of injecting a mouse iPS cell into a rat embryo and a method of injecting a rat iPS cell into a mouse embryo were carried out.

(1) Establishment of Rat iPS Cell Having Ability to Form Chimera

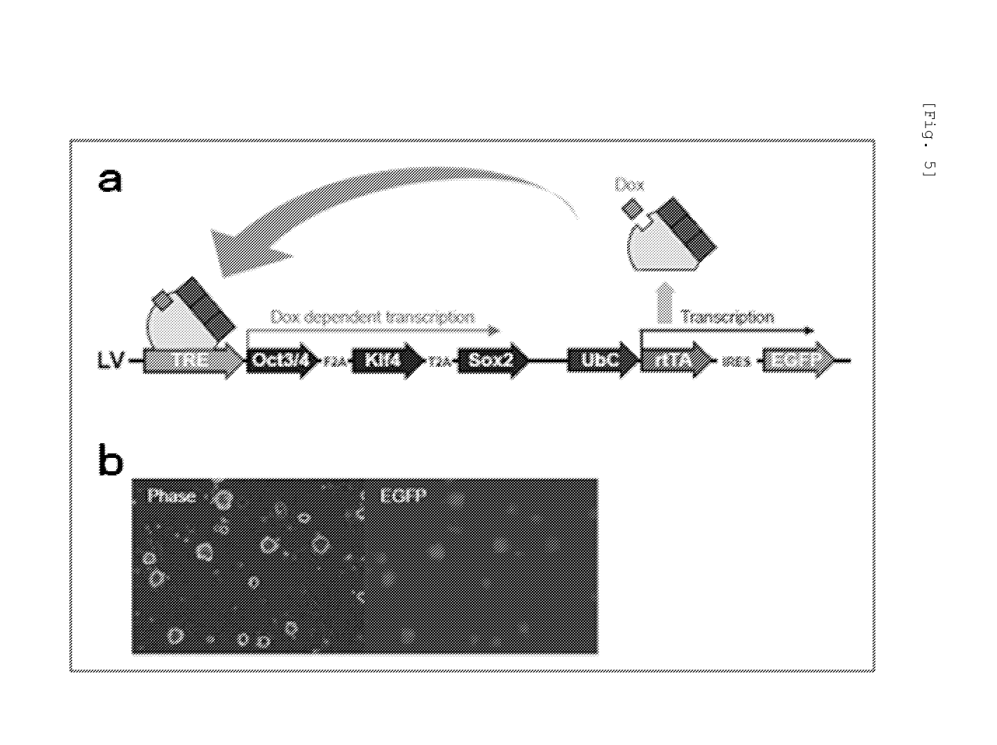

A fibroblast was established from a Wistar rat fetus (male) that served as the source of iPS cell establishment. For the induction of the iPS cell, not a retrovirus used in establishing the mouse iPS cell but a lentivirus vector incorporating a system capable of inducing expression of 3 factors tetracycline-dependently was used (FIG. 5a). With this vector, both rtTA and EGFP are expressed through the IRES sequence under the ubiquitin-C (UbC) promoter. Accordingly, a cell found to be infected with the lentivirus during the induction constantly shows EGFP fluorescence, enabling labeling of the established iPS cell itself also. The 3 factors were introduced through this lentivirus, and the rat iPS cell was induced. After the introduction, culture was performed for a while on a medium containing a serum necessary for growth of the fibroblast. Then, in the middle, the culture was continued after replaced with a rat ES cell medium containing an inhibitor. As a result, rat iPS cell-like colonies were obtained. In subculturing after the pick-up also, the colonies were able to be maintained without spontaneous differentiation, and iPS cell lines were successfully established. One of the cell lines thus established is a rat iPS cell (rWEi3.3-iPS cell) (FIG. 5b).

Note that the culture conditions for the iPS cell are as follow. For the maintenance of the undifferentiation of the mouse iPS cell, a medium in which 15% knockout serum replacement (Invitrogen Corp.), 0.1 mM 2-mercaptoethanol, 0.1 mM non-essential amino acid, 1 mM HEPES buffer solution (Invitrogen Corp.), 1% L-glutamine penicillin streptomycin, and 1000 U/ml mouse LIF were added to Dulbecco's modified Eagle's medium (Sigma Corporation) was used and spread on the mouse embryonic fibroblast having been subjected to mitomycin-C treatment.

For the maintenance of the undifferentiation of the rat iPS cell, a medium in which 1 μM MEK inhibitor PD0325901 (Axon, Groeningen, Netherland), 3 μM GSK3 inhibitor CHIR99021 (Axon), FGF receptor inhibitor SU5402 (Calbiochem, La Jolla, Calif.), and 1000 U/ml rat LIF (Millipore) were added to an N2B27 medium (Ying et al., 2003) was used and spread on the mouse embryonic fibroblast having been subjected to mitomycin-C treatment.

Any of the iPS cells were cultured in the presence of 5% CO₂ at 37° C.

(2) Analysis of Mouse-Rat Heterologous Chimera in Fetal Period

To produce a heterologous chimera, a mouse iPS cell was injected into a rat blastocyst, or reversely a rat iPS cell was injected into a mouse blastocyst. As the mouse iPS cell, an mGT3.2-iPS cell derived from EGFP-Tg mouse established in Example 1 was used. The embryo culture and the embryo manipulation are as follow. Collection, culture, microinjection, and so forth of an embryo after mice were bred were carried out according to the published protocol (Nagy et al., 2003). In a brief description, first, mouse 8-cell stage/morula embryos from the oviduct and uterus 2.5 days after breeding were collected into M2 medium (Millipore). The collected embryos were transferred into a KSOM medium containing an amino acid (KSOM-AA medium: Millipore), and cultured in the presence of 5% CO₂ at 37° C. for 1 to 2 hours until the embryos were used for injection manipulation in a case of use for injection into the 8-cell stage/morula embryo, or overnight in a case of use for injection into the blastocyst.

The rat 8-cell stage/morula embryos were collected from the oviduct and uterus 3.5 days after breeding, and the blastocysts were collected from the uterus 4.5 days after breeding, into a HER medium (Ogawa et al., 1971). The collected embryos were transferred into an mR1ECM medium (Oh et al., 1998) and cultured in the presence of 5% CO₂ at 37° C. for 1 to 2 hours until the embryos were used for injection manipulation in a case of use for injection into the embryo.

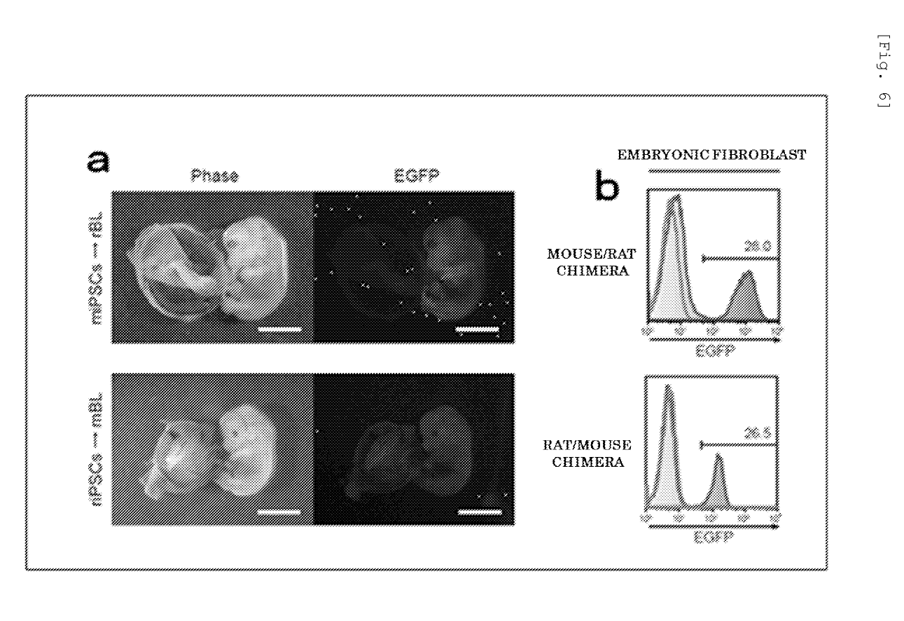

For micromanipulation, the iPS cells were detached from the dish by 0.25% trypsin/EDTA (Invitrogen Corp.) treatment and suspended in each medium. Using a piezo-micromanipulator (Primetech, Tokyo, Japan), pores were created in the zona pellucida and trophectoderm of the embryo under a microscope, and approximately 10 iPS cells were injected into the perivitelline space of the 8-cell stage/morula embryo or near the inner cell mass of the blastocyst. After the injection, the 8-cell stage/morula embryo was cultured into the blastocyst stage overnight, and the blastocyst was cultured for 1 to 2 hours, in each medium, and then the embryo was transplanted. The rat blastocyst was transplanted into the uterus of a pseudo-pregnant rat, while the mouse blastocyst was transplanted into the uterus of a pseudo-pregnant mouse. As the transplanted recipient, an ICR strain female mouse on day 2.5 after crossing with a vasoligated male mouse was used in a case of using the mouse embryo, while a Wistar strain female rat on day 3.5 after crossing with a vasoligated rat was used in a case of using the rat embryo. Since the period to reach the full term pregnancy was longer for rat by 2 days, the analysis timing was set differently between the two by 2 days, so that the fetuses approximately in the same stage were analyzed. Thus, the analysis timing was on embryonic day 15 in a case where the rat was the host embryo, and on embryonic day 13 in a case where the mouse was the host embryo. As a result of the analysis, EGFP fluorescence was observed from the rat fetus injected with the mouse iPS cell, and vice versa (FIG. 6a). Fibroblasts were established from the obtained fetuses and analyzed using a flow cytometer. Peaks of EGFP positive were observed from both of the chimeras. This result supported the production of a chimera contributed by the iPS cell (FIG. 6b).

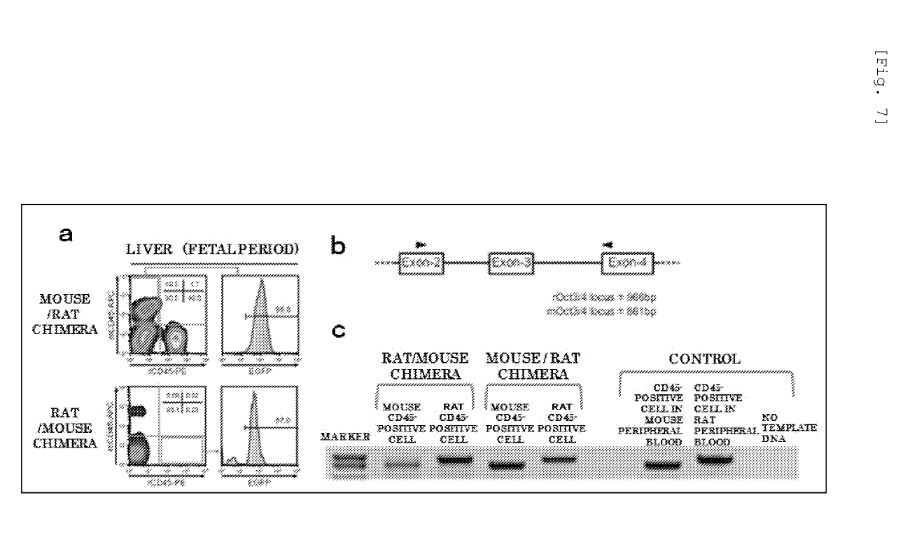

Moreover, livers were taken out from the same fetuses and analyzed with a flow cytometer after blood systems were specifically stained with CD45 antibodies respectively specific to mouse and rat (APC-labeled anti-mouse CD45 antibody, PE-labeled anti-rat CD45 antibody). As a result, sole fractions of a mouse CD45-positive cell and a rat CD45-positive cell respectively existed in the fetal livers (FIG. 7a).

Further, the mouse and rat CD45-positive cells were respectively sorted, and genomic DNA was extracted to examine the genetic origins of the two, and PCR was carried out. For genotyping between the heterologous species, DNA extracted using QIAamp DNA Mini Kit was used, and the following primer set was used for PCR.

mouse and rat Oct3/4

### Fw: 5′-CAGTTTGCCAAGCTGCTGAA-3′ (SEQ ID NO: 10)

### Rv: 5′-AGGGTCTCCGATTTGCATAT-3′ (SEQ ID NO: 11)

For determination of the origins, attention was focused on the difference in intron length between exon 2 and exon 4 at the Oct3/4 locus in both of the genomic DNAs. The length of this portion of a rat is longer by approximately 100 bases. By performing PCR with a primer designed based on the common sequence present on the exons, the origins of the two can be determined (FIG. 7b). As a result of PCR, a length of the mouse Oct3/4 locus was shown for the genomic DNA of the mouse CD45-positive cell, and a length of the rat Oct3/4 locus was shown for the rat CD45-positive cell (FIG. 7c). Accordingly, from the analysis result using the surface antigen, or also from the genetic analysis result, it was found that both the cells existed chimera fetal liver. From the above, the production of a heterologous chimera between mouse and rat was proved.

(3) Growth of Mouse-Rat Heterologous Chimera into Neonate and Adult

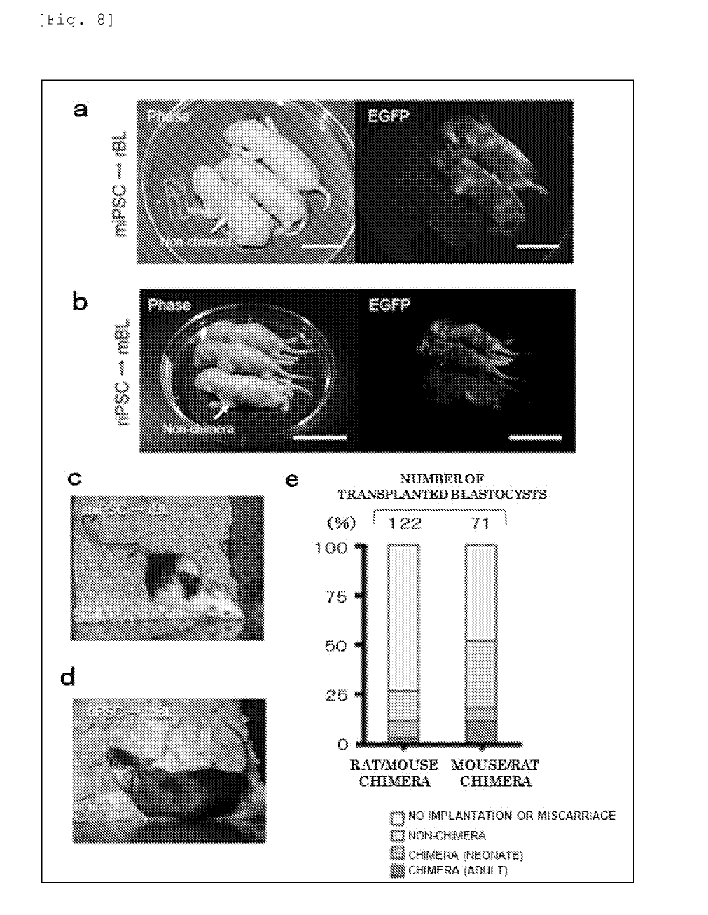

Next, we examined whether or not the mouse-rat heterologous chimera could grow into the full term pregnancy or adult after birth. In this method, the mouse mGT3.2-iPS cell used in the previous section was injected into a rat blastocyst, while the rat rWEi3.3-iPS cell was injected into a mouse blastocyst. After transplantation into the uteruses, litters were taken out in the full term pregnancy by natural delivery or Caesarean section. As a result of observation of the neonates under a fluorescent microscope, both of the litters showed mosaic EGFP fluorescence (FIGS. 8a, 8b). In addition, the litters were grown into adults after birth, and chimera formation was judged from coat color. Since derived from an EGFP-Tg mouse having the black-haired C57BL/6 strain background, the mouse iPS cell was distinguishable from one derived from a white-haired Wistar rat embryo. Reversely, since derived from white-haired Wistar, the rat iPS cell was distinguishable from one derived from an F1 embryo of black-haired C57BL/6×BDF1. As a result, heterologous chimera formation was confirmed in adult also (FIGS. 8c, 8d).

From the above, it was found that the development of the heterologous chimera proceeded into the full term pregnancy and grew normally after birth, as well (FIG. 8e).

Further, the chimerism of the heterologous chimera was determined. In this method, a peripheral blood after hemolysis in the living body was stained with an APC-labeled anti-mouse CD45 antibody, a PE-labeled anti-rat CD45 antibody, a PE-labeled anti-Gr-1 antibody, a PE-labeled anti-Mac-1 antibody (rat IgG: BD Bioscience Pharmingen), a PE-Cy7-labeled anti-B220 antibody (rat IgG: BD Bioscience Pharmingen), an APC-labeled anti-CD4 antibody, and an APC-Cy7-labeled anti-CD8 antibody. Then, sorting and analysis were performed using Mo-flo and FACSCanto (BD bioscience).

(4) Origin of Cell in Each Tissue of Heterologous Chimera

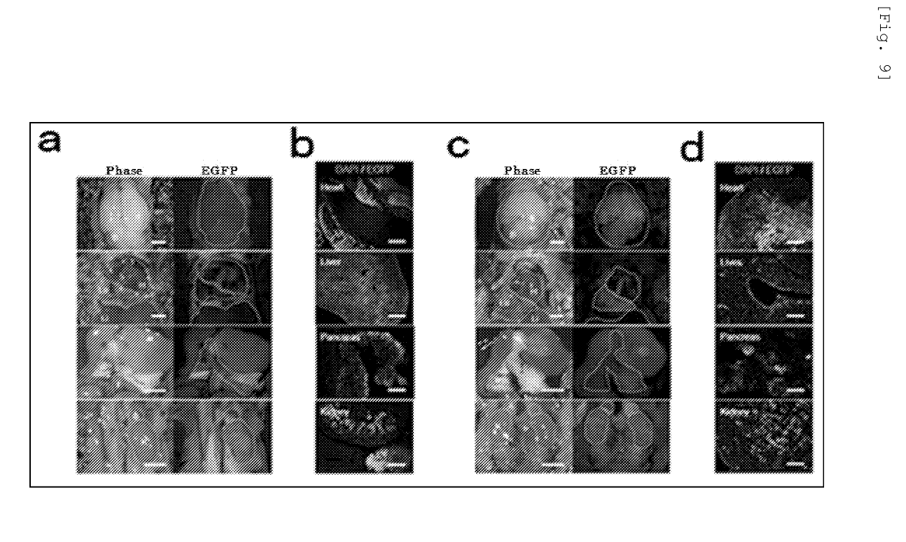

The neonate individuals were analyzed under a fluorescence stereoscopic microscope to what degree the heterologous iPS cell-derived cells contribute in the chimera individuals. EGFP-positive mouse iPS cell-derived cells were observed in almost all the organs of the heterologous chimera individual obtained by injecting the mouse iPS cell into the rat blastocyst (FIG. 9a). Tissue sections were prepared from representative organs (heart, liver, pancreas, kidney), and localization of the mouse iPS cell-derived cells were examined using an anti-EGFP antibody. As a result, mosaic EGFP fluorescence was observed in all the tissues, and chimerization was found out (FIG. 9b). Moreover, a similar result was obtained in the heterologous chimera individual obtained by injecting the rat iPS cell into the mouse blastocyst (FIGS. 9c and 9d). This suggests that both of the iPS cells are capable of differentiation into almost all the tissues and organs in the entire body even in a heterologous environment.

From the above, it is suggested that a heterologous chimera can be produced using an iPS cell, and that the injected iPS cell can be differentiated into a functional cell in the entire body even in a heterologous environment through normal embryogenesis.

### Example 3

Based on the techniques for producing a knockout rat targeting organ deficiency through establishment of rat ES cells and iPS cells in recent reports (for example, Cell Stem Cell 4, 16-19, Dec. 18, 2008=Rat iPS Cell 135, 1287-1298, Dec. 26, 2008=Rat ES Cell 135, 1299-1310, Dec. 26, 2008=RatES, and so on), these knockout rats are used as a host.

In accordance with Example 1, a mouse stem cell is then injected into the host to produce a chimeric animal. As a result, a heterologous organ regenerated for the knockout organ can be observed.

### Example 4

Moreover, on the other hand, by injecting a rat stem cell line into an existing organ deficient mouse in reverse to Example 3, a similar effect can be obtained. In the experiment, a chimera is produced in accordance with Example 1, and then a heterologous organ regenerated for the knockout organ can be observed.

As described above, the present invention has been illustrated using preferred embodiments of the present invention, but it is understood that the scope of the present invention should be construed only by the claims. It is understood that the patents, patent applications, and articles cited herein should be such that the disclosures thereof should be incorporated into the present description by reference, as with the disclosures themselves are specifically described in the present description.

### INDUSTRIAL APPLICABILITY

The present invention provides a fundamental technique that greatly facilitates creation of useful animal species in the field of animal engineering, improvement in livestock, and so forth. Moreover, the present invention provides a technique also applicable to a technique for regenerating an “own organ” from a somatic cell, such as skin, depending on the circumstance of an individual.

1. A method for producing a heterologous chimeric animal, comprising steps:
(A) injecting an induced pluripotent stem (iPS) cell into a blastocyst cavity in a blastocyst stage of an animal heterologous to that of the iPS cell; and
(B) transplanting the blastocyst injected with the iPS cell in the step (A) into a womb of a non-human host mammal that is the heterologous animal; growing the blastocyst; and thereby obtaining a litter that is a chimeric animal between a species of the iPS cell and a species of the heterologous animal.

2-20. (canceled)


SEQUENCE LISTING (11 sequences)

SEQ ID NO: 1 - DNA, 24 bp, Artificial
  primer
ccctggggatgctgtgagccaagg

SEQ ID NO: 2 - DNA, 24 bp, Artificial
  primer
ccctttttctggagactaaataaa

SEQ ID NO: 3 - DNA, 24 bp, Artificial
  primer
gcgaactcacacaggcgagaaacc

SEQ ID NO: 4 - DNA, 24 bp, Artificial
  primer
ttatcgtcgaccactgtgctgctg

SEQ ID NO: 5 - DNA, 24 bp, Artificial
  primer
ggttacctcttcctcccactccag

SEQ ID NO: 6 - DNA, 24 bp, Artificial
  primer
cagaggaggaacgagctgaagcgc

SEQ ID NO: 7 - DNA, 21 bp, Artificial
  primer
ttcatgcgacggttttggaac

SEQ ID NO: 8 - DNA, 21 bp, Artificial
  primer
ttcaacatcactgccagctcc

SEQ ID NO: 9 - DNA, 18 bp, Artificial
  primer
tgtgagcgagtaacaacc

SEQ ID NO: 10 - DNA, 20 bp, Artificial
  primer
cagtttgccaagctgctgaa

SEQ ID NO: 11 - DNA, 20 bp, Artificial
  primer
agggtctccgatttgcatat